(19) United States
(12) Patent Application Publication
(10) Pub. No.: US 2024/0226462 A1
(43) Pub. Date: Jul. 11, 2024

(54) DROPLET DELIVERY DEVICE WITH HIGH DOSE CONFIDENCE MODE

(71) Applicant: PNEUMA RESPIRATORY, INC., Boone, NC (US)

(72) Inventors: Charles Eric Hunter, Boone, NC (US); Gregory Rapp, Boone, NC (US)

(73) Assignee: PNEUMA RESPIRATORY, INC., Boone, NC (US)

(21) Appl. No.: 18/408,349

(22) Filed: Jan. 9, 2024

Related U.S. Application Data
(60) Provisional application No. 63/437,909, filed on Jan. 9, 2023.

(51) Int. Cl. A61M 11/00

(52) U.S. Cl. CPC A61M 11/005; A61M 2205/18; A61M 2205/33

(57) ABSTRACT

A droplet delivery device includes a high dose confidence mode that monitors a user's inhalation of a composition and activates and deactivates aerosol generation via monitoring the user's inhalation to assist the user to complete a full dosage when a user does not inhale a full dosage of the composition when using the device.

### CROSS-REFERENCE TO RELATED APPLICATIONS

This application claims the benefit of priority of U.S. Provisional Application No. 63/437,909, entitled “Droplet Delivery Device with High Dose Confidence Mode,” filed Jan. 9, 2023, which is incorporated herein by reference in its entirety.

### FIELD OF THE INVENTION

This disclosure relates to droplet delivery devices and more specifically to droplet delivery devices for the delivery of fluids that are inhaled into mouth, throat, nose, and/or lungs. While the present invention particularly pertains to the delivery of therapeutic compositions, in some embodiments non-therapeutic compositions that are capable of being aerosolized could also be used.

### BACKGROUND

The present application incorporates herein by reference in their entirety the contents of WO 2020/264501 (describing “Ring Mode” ejection), PCT/US2022/034552 (describing “Push Mode” ejection), U.S. Pat. No. 10,449,314 (describing dose verification) and US Patent Application Pub. No. 20190134330 (describing user feedback and instructions).

There is a need for droplet delivery devices, such as those devices described in Applicant's referenced patent publications, to measure dosage throughout the administration/inhalation process of a patient to provide a high dose confidence mode of proper dosage being administered to the patient. Specifically, the present invention can determine if a patient does not receive the full dose initially and the device calculates the remaining dosage and gives the patient a voice command, visual notification, or similar communication to inhale again to complete the dosage.

### SUMMARY OF THE INVENTION

Droplet delivery devices of the present invention, such as for inhaling medicines, pharmaceuticals, biologics, vaccines, and other therapeutic substances, use a differential pressure sensor to measure the user's inhalation profile over the course of a dosage. In some embodiments, a Sensirion SDP3x™ sensor (available from Sensirion AG located at Laubisruetistrasse 50, 8712 Stsfa, Switzerland, with a U.S. location/affiliate of Sensirion Connected Solutions Inc., 11 East Adams Suite 220, Chicago, IL 60603), which has a pressure update rate of 0.5 milliseconds and accuracy of 0.1 Pa and is included in a droplet delivery device to measure the user's inhalation profile near real time.

In an embodiment, a user initiates their dosage by inhaling from the mouthpiece of a droplet deliver device. Upon inhalation, the device begins actuating an ejector member, such as piezoelectric element/transducer in combination with a mesh or plate with apertures to generate aerosol—such as a piezoelectric transducer in “Push Mode” ejection devices and piezoelectric elements in “Ring Mode” ejection devices (disclosed by incorporation by reference herein). If the patient stops inhaling from the mouthpiece before their metered dosage is complete, the device will stop generating aerosol. The device will then wait until the patient is inhaling from the mouthpiece to deliver the remaining dosage.

For example, if a user's dosage requires a 1.5 second inhalation and they stop inhaling halfway at 0.75 seconds, they still need to take the other half of their dosage. Droplet devices of the present invention sense that a remaining amount of dosage is yet to be completed and stop generating aerosol then prompt the user that they did not receive their entire dosage and instruct them to inhale from the mouthpiece again. When the user initiates the spray for the second time, the device will begin to generate aerosol and continue until the other half of their dosage is inhaled. It will be appreciated that remaining dosage and aerosol generation of time for the remaining amount of dosage can be any difference amount between a full dosage and the amount the user inhaled (e.g., based on time) during the initial administration event.

### BRIEF DESCRIPTION OF THE DRAWINGS

FIG. 1 illustrates a relational block diagram of a droplet device components and microcontroller unit (MCU) in one embodiment of the present invention.

### DETAILED DESCRIPTION

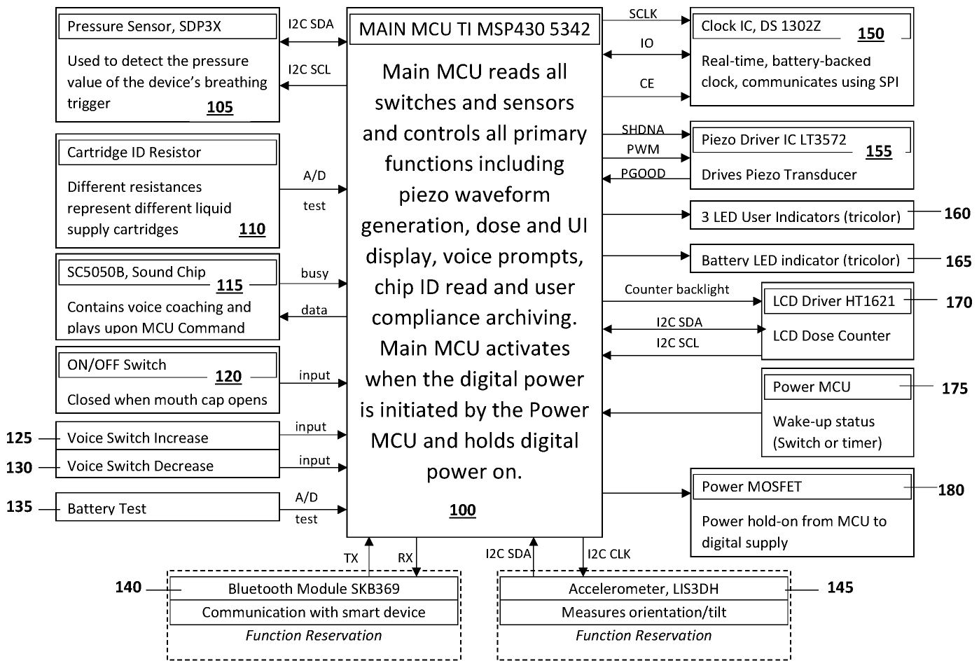

With reference to FIG. 1, the MCU 100 reads all switches and sensors and controls all primary functions including piezo waveform generation, dose and user interface (UI) display, voice prompts, chip ID read and user compliance archiving. Main MCU 100 activates when the digital power is initiated by the Power MCU 175 and holds digital power on. On/Off Switch 120 may be closed when the mouth cap opens to initiate power and readiness of the device to provide aerosol generation. A battery test 135 may also be operably connected to the MCU 100.

The total inhalation time is determined by the drug application. Droplet devices of the present invention are metered to a specific drug dispense rate, and depending on that parameter the total inhalation time may be determined. The predetermined dosage time is then specified in the software of the microcontroller to provide the high dose confidence mode. Cartridge ID resistor 110 may provide different resistances to identify different liquid supply cartridges so that the corresponding dosage, including dispense rate and inhalation time, is determined for the corresponding drug application.

In some embodiments, drug efficacy can be calculated through some combination of the instantaneous flow rate from the user and the amount of time inhaled. For example, if a user was inhaling at sufficient flow rate for the full dosage time the drug efficacy would be 100%. If the user was inhaling at a less than sufficient flow rate for the full dosage time the drug efficacy less than 100%. If the user was inhaling at a sufficient flow rate for half the dosage time the drug efficacy would be 50%. The device could then allow for a successive administration to deliver the remaining portion of their dosage.

The timing of the start/stop method is a timer program of the microcontroller based on the pressure change measured from the pressure sensor 105. The droplet delivery device begins actuating the piezoelectric element/transducer with piezo driver IC 155 and starts a timer via clock IC 150 when the system notices a drop in pressure from a user inhaling on the mouthpiece and sense by pressure sensor 105. The device then stops the timer via clock IC 150 and stops piezo element/transducer actuation after the pressure sensor value sensed from sensor 105 drops below an accepted value. The delay from when the user starts inhaling to the device actuating the spray can be less than or equal to 0.5 milliseconds due to the measurement time on the Sensirion™ pressure sensor 105. The delay from when the dosage timer starts to when the piezoelectric is actuated is much less, on the scale of microseconds.

The pressure sensor 105 may be calibrated, for example, by using the standard 3 L syringe for spirometry described at https://www.a-msystems.com/p-60-fixed-volume-calibration-syringe.aspx (incorporated herein by reference). The start/stop condition may be triggered by any of the following (which could be useful to ensure drug delivery to specific sections of the lung):
(1) Instantaneous pressure threshold—Either the user stops inhaling entirely or the user could not be inhaling with a sufficient flow rate; and
(2) A threshold for the volume of air or some other calculated value could be used for the start/stop condition.

In some embodiments, a communication or notification may be provided to the user that there is still a remaining amount of dosage necessary to reach full dosage. The user may be prompted, such as by an audio speaker communicating with sound chip 115 or visual notification, to again inhale on the device to finish administering the dosage. Voice switch increase 125 and voice switch decrease 130 inputs permit the user to adjust volume of the audio from the speaker. Visual notifications to communicate user actions and device readiness are provided to the user by LED indicators 160, such as tricolor LEDs, and power status is signified by a battery LED indicator 165. An LCD Driver 170 may display a dose counter or similar indications of dosage administered and/or remaining to the user.

The piezo element/transducer is subsequently actuated for the remaining amount of dosage time (i.e., delivering the remaining amount to reach full dosage based on the calculated time that is remaining to reach full dosage) on detection of another pressure drop from the pressure sensor when the user inhales to complete the dosage. The microcontroller will deactivate the piezoelectric element after the remaining duration of inhalation to reach full dosage is completed by user.

The trickle-charge real time clock circuit 150 is important for Internet Of Things (IOT) applications (with paired mobile/desktop application such as via Bluetooth module 140) as it would enable each dosage to be accurately tracked in real time. This circuit can use a very small amount of power to keep a clock running on the device even while the device is completely powered off.

There are at least two alternatives that can be used to drive the piezo element/transducer. A first alternative is that the piezoelectric element/transducer is driven by a waveform generated by a boost converter, H-bridge circuit. The second alternative is an autotransformer and MOSFET circuit 180. These both work similarly to one another in that a frequency generated by the microcontroller pulse width modulation or a separate integrated circuit determines the frequency of the waveform applied to the piezo element/transducer. The alternatives differ in how they generate high voltage (>30V). The ejection start, or actuation of the piezoelectric element/transducer, is initiated by the microcontroller in software by either sending a PWM to a MOSFET or I2C signal to a frequency generator chip. The ejection stop, or deactivation of the piezoelectric element/transducer, is initiated by either stopping the PWM to a MOSFET or send another I2C signal to the frequency generator chip.

A Bluetooth module 140 may be provided in embodiments to permit communication with smart devices, such as smartphones, for tracking dosage administration and/or other data associated with the user and device via a Bluetooth-enabled application running on the smart device. Data received by the application may be communicated to a network (such as via an Internet connection), including processing such data to provide resulting messages and information about the user's treatments, compliance, dosage, and use of the device to a healthcare provider or patient/individual monitoring service. An accelerometer 145 may also be operable connected to the MCU 100 to provide orientation and tilting information for the device that might could necessitate change the dosage calculations or trigger notification to the user to adjust how the device is positioned to optimize the delivery of a drug application.

While piezotronics are described in embodiments of the invention, including specific types and brands of electronics, it will be appreciated that other elements may be utilized as transducers to provide vibrating functionality to an ejector for aerosol generation. Similarly, although specific data communication protocols (I2C, SPI, etc) are described or illustrated in certain embodiment of the invention, it will be appreciated by those skilled in the relevant art that a variety of other data communication protocols could be applied with the same effect.

Various embodiments of the invention have been described. It will, however, be evident that various modifications and changes may be made thereto, and additional embodiments may be implemented, without departing from the broader scope of the invention as set forth by the disclosure. This specification is to be regarded in an illustrative rather than a restrictive sense.

What is claimed:

1. A droplet delivery device comprising:
a vibrating ejector member coupled to a supply of a composition; and
a dosage monitor that powers the vibrating ejector member on and off based on a user's incomplete inhalation of a full dosage of the composition.

2. The device of claim 1, further comprising a pressure sensor in communication with a microcontroller of the device, wherein the microcontroller turns the vibrating ejector member on and off based on a pressure value of a user's inhalation relative to the device.

3. The device of claim 2, further comprising a speaker in communication with the microcontroller to provide audible notification to the user of a remaining dosage necessary to complete administration of the full dosage of the composition.

4. The device of claim 3, further comprising a display or light in communication with the microcontroller to provide visual notification to the user of a remaining dosage necessary to complete administration of the full dosage of the composition.

5. The device of claim 2, further comprising a display or light in communication with the microcontroller to provide visual notification to the user of a remaining dosage necessary to complete administration of the full dosage of the composition.

6. The device of claim 1, further comprising a liquid supply cartridge having an identifier that communicates the full dosage parameters for the composition corresponding to the liquid supply cartridge to the dosage monitor.

7. The device of claim 2, further comprising a liquid supply cartridge having an identifier that communicates the full dosage parameters for the composition corresponding to the liquid supply cartridge to the microcontroller.

8. The device of claim 3, further comprising a liquid supply cartridge having an identifier that communicates the full dosage parameters for the composition corresponding to the liquid supply cartridge to the microcontroller.

9. The device of claim 4, further comprising a liquid supply cartridge having an identifier that communicates the full dosage parameters for the composition corresponding to the liquid supply cartridge to the microcontroller.

10. The device of claim 5, further comprising a liquid supply cartridge having an identifier that communicates the full dosage parameters for the composition corresponding to the liquid supply cartridge to the microcontroller.

11. The device of claim 2, wherein the microcontroller is programmed to calculate drug efficacy and determine a remaining dosage necessary to complete administration of the full dosage of the composition by detecting from the pressure sensor during the user's inhalation a combination of instantaneous flow rate and amount of time inhaled.

12. The device of claim 3, wherein the microcontroller is programmed to calculate drug efficacy and determine a remaining dosage necessary to complete administration of the full dosage of the composition by detecting from the pressure sensor during the user's inhalation a combination of instantaneous flow rate and amount of time inhaled.

13. The device of claim 4, wherein the microcontroller is programmed to calculate drug efficacy and determine a remaining dosage necessary to complete administration of the full dosage of the composition by detecting from the pressure sensor during the user's inhalation a combination of instantaneous flow rate and amount of time inhaled.

14. The device of claim 5, wherein the microcontroller is programmed to calculate drug efficacy and determine a remaining dosage necessary to complete administration of the full dosage of the composition by detecting from the pressure sensor during the user's inhalation a combination of instantaneous flow rate and amount of time inhaled.

15. The device of claim 7, wherein the microcontroller is programmed to calculate drug efficacy and determine a remaining dosage necessary to complete administration of the full dosage of the composition by detecting from the pressure sensor during the user's inhalation a combination of instantaneous flow rate and amount of time inhaled.

16. The device of claim 8, wherein the microcontroller is programmed to calculate drug efficacy and determine a remaining dosage necessary to complete administration of the full dosage of the composition by detecting from the pressure sensor during the user's inhalation a combination of instantaneous flow rate and amount of time inhaled.

17. The device of claim 9, further comprising a liquid supply cartridge having an identifier that communicates the full dosage parameters for the composition corresponding to the liquid supply cartridge to the microcontroller.

18. The device of claim 10, further comprising a liquid supply cartridge having an identifier that communicates the full dosage parameters for the composition corresponding to the liquid supply cartridge to the microcontroller.

19. A droplet delivery device comprising:
a vibrating ejector member coupled to a supply of a composition;
a transducer coupled to the vibrating ejector member; and
a microcontroller that powers the transducer on and off based on a user's incomplete inhalation of a full dosage of the composition.

20. The device of claim 19, further comprising a pressure sensor in communication with the microcontroller, wherein the microcontroller automatically turns the transducer on and off based on a pressure value of a user's inhalation relative to the device.
